(19) United States
(12) Patent Application Publication
(10) Pub. No.: US 2021/0001020 A1
(43) Pub. Date: Jan. 7, 2021

(54) Manual Pump With Charge Capability

(71) Applicant: KCI Licensing, Inc., San Antonio, TX (US)

(72) Inventor: Christopher Brian LOCKE, Bournemouth (GB)

(21) Appl. No.: 16/961,983

(22) PCT Filed: Mar. 8, 2019
(86) PCT No.: PCT/US2019/021353

§ 371 Date: Jul. 14, 2020

Related U.S. Application Data
(60) Provisional application No. 62/650,752, filed on Mar. 30, 2018.

(51) Int. Cl. A61M 1/00

(52) U.S. Cl. CPC A61M 1/0009; A61M 1/0041; A61M 1/0088; A61M 2205/071; A61M 1/0072

(57) ABSTRACT

An apparatus for charging a negative-pressure source is described. The apparatus includes a body having a first end, a second end, a suction chamber, and a longitudinal axis. A slider at least partially surrounds the body and is configured to slide relative to the body parallel to the longitudinal axis. A piston is disposed in the suction chamber and slidable relative to the body parallel to the longitudinal axis. The apparatus includes a rod having a first end coupled to the slider and a second end coupled to the piston. The first end and the second end can move parallel to the longitudinal axis in opposite directions. An end cap is coupled to the body. The end cap is configured to locate at least one constant force spring and turn the rod from the first direction to the second direction.

### RELATED APPLICATIONS

This application claims the benefit, under 35 U.S.C. § 119(e), of the filing of U.S. Provisional Patent Application Ser. No. 62/650,752, entitled “MANUAL PUMP WITH CHARGE CAPABILITY,” filed Mar. 30, 2018, which is incorporated herein by reference for all purposes.

### TECHNICAL FIELD

The invention set forth in the appended claims relates generally to tissue treatment systems and more particularly, but without limitation, to a manually operated pump with charging capability.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for charging a negative pressure device in a negative-pressure therapy environment are set forth in the appended claims. Illustrative embodiments are also provided to enable a person skilled in the art to make and use the claimed subject matter.

For example, an apparatus for charging a negative-pressure source may be described. The apparatus can include a body having a first end, a second end, a suction chamber, and a longitudinal axis. A slider can at least partially surround the body and be configured to slide relative to the body parallel to the longitudinal axis. A piston can be disposed in the suction chamber and configured to slide relative to the body parallel to the longitudinal axis. A rod having a first end can be coupled to the slider and a second end can be coupled to the piston. The first end may be configured to move parallel to the longitudinal axis in a first direction, and the second end may be configured to move parallel to the longitudinal axis in a second direction. An end cap can be coupled to the first end of the body. The end cap may be configured to locate at least one constant force spring and turn the rod from the first direction to the second direction.

In some embodiments, the slider further can further comprise a ring. In some embodiments, the slider further can comprise two sliders. In some embodiments, each of the two sliders can further comprise a finger location. In some embodiments, the first direction is opposite the second direction. In some embodiments, the rod can be an injection molded polymer construction. In other embodiments, the rod can be formed from TPE or silicone. In some embodiments, the rod may have a shore rating of about Shore 60 to about Shore 90. The rod is can be configured to deform, forming a radius and transmitting a linear force through the radius. The rod can further include slits along a length of the rod, the slits configured to facilitate bending and the linear transmission of force. Sometimes, the rod further comprises a plurality of grooves across a width of the rod. The rod can be two rods, each having a plurality of grooves, the grooves of each rod configured to mesh with each other, thereby coupling the two rods.

More generally, a system for generating negative pressure may be described. The system can include a pump housing having a first end, a second end, a chamber, and a longitudinal axis. A tensioner can at least partially surround the pump housing and be configured to slide relative to the pump housing parallel to the longitudinal axis. A head can be disposed in the chamber and configured to slide relative to the pump housing parallel to the longitudinal axis. A connector having a first end may be coupled to the tensioner and a second end may be coupled to the head. The first end can be configured to move parallel to the longitudinal axis in a first direction, and the second end can be configured to move parallel to the longitudinal axis in a second direction. An end cap can be coupled to the first end of the pump housing and configured to locate at least one constant force spring and turn the connector 180 degrees. A nozzle may be coupled to the second end of the pump housing and fluidly coupled to the chamber. The nozzle can be configured to be fluidly coupled to a tissue site.

In some embodiments, the tensioner may be configured to move in the second direction, sliding the head in the first direction within the chamber. In other embodiments, the connector may be configured to deform, forming a radius and transmitting a linear force through the radius. The first direction may be opposite the second direction. The pump housing can further comprise an elliptical tube having the chamber disposed within the tube. At least one groove may be formed in an exterior of the elliptical tube. The at least one groove can be configured to receive the connector, and the connector can be configured to slide relative to the elliptical tube through the groove.

Alternatively, other example embodiments may describe a method for generating negative pressure. A negative-pressure source can be provided. The negative pressure source can include a body having a first end, a second end, a suction chamber, and a longitudinal axis. A slider can at least partially surround the body and be configured to slide relative to the body parallel to the longitudinal axis. A piston can be disposed in the suction chamber and configured to slide relative to the body parallel to the longitudinal axis. A rod having a first end can be coupled to the slider and a second end can be coupled to the piston. The first end may be configured to move parallel to the longitudinal axis in a first direction, and the second end may be configured to move parallel to the longitudinal axis in a second direction. An end cap can be coupled to the first end of the body. The end cap may be configured to locate at least one constant force spring and turn the rod from the first direction to the second direction. The slider can be moved in the first direction, thereby moving the piston in the second direction and tensioning the constant force spring.

An apparatus for charging a negative-pressure source is also described herein. The apparatus can include a body having a first end, a second end, a suction chamber, and a longitudinal axis. A piston can be disposed in the suction chamber and configured to slide relative to the body parallel to the longitudinal axis. A slider can at least partially surround the body. The slider can be coupled to the piston and configured to move parallel to the longitudinal axis in a first direction. Movement of the slider in the first direction can cause the piston to move parallel to the longitudinal axis in a second direction. In some embodiments, the slider can further comprise a ring. In some embodiments, the slider further comprises two sliders. The first direction can be opposite the second direction.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a simplified functional block diagram of an example embodiment of a therapy system that can provide negative-pressure therapy to a tissue site in accordance with this specification;

FIG. 2A is a perspective exploded view of a therapy unit of FIG. 1 illustrating additional details that may be associated with some example embodiments of the therapy system of FIG. 1;

FIG. 2B is a detail end view of a portion of the therapy unit of FIG. 2A illustrating additional details that may be associated with some example embodiments of the therapy system of FIG. 1;

FIG. 3 is a perspective exploded view of the therapy unit illustrating additional details that may be associated with some example embodiments of the therapy system of FIG. 1;

FIG. 4 is a perspective view of the therapy unit illustrating additional details that may be associated with some example embodiments of the therapy system of FIG. 1;

FIG. 5 is a perspective top assembly view of a spring assembly of the therapy unit illustrating additional details that may be associated with some example embodiments of the therapy system of FIG. 1;

FIG. 6 is a perspective bottom assembly view of the spring assembly illustrating additional details that may be associated with some example embodiments of the therapy system of FIG. 1;

FIG. 7 is a perspective top assembly view of a piston assembly of the therapy unit illustrating additional details that may be associated with some example embodiments of the therapy system of FIG. 1;

FIG. 8 is a perspective bottom assembly view of the piston assembly illustrating additional details that may be associated with some example embodiments of the therapy system of FIG. 1;

FIG. 9 is a perspective view of a charging assembly of the therapy unit illustrating additional details that may be associated with the therapy system of FIG. 1;

FIG. 10 is an end view of an end cap, and the charging assembly illustrating additional details that may be associated with some embodiments of the therapy system of FIG. 1;

FIG. 11 is a section view of the end cap and the charging assembly taken along line 11-11 of FIG. 10 and illustrating additional details that may be associated with some embodiments of the therapy system of FIG. 1;

FIG. 12 is an assembled sectional view of the end cap, the spring assembly, the piston assembly, and the charging assembly illustrating additional details that may be associated with the therapy system of FIG. 1;

FIG. 13 is a perspective view of the spring assembly, the piston assembly, and the charging assembly illustrating additional details that may be associated with the therapy system of FIG. 1;

FIG. 14 is a perspective assembly view of the spring assembly, the piston assembly, and the charging assembly illustrating additional details that may be associated with some embodiments of the therapy system of FIG. 1;

FIG. 15A is a front view of the therapy unit illustrating additional details that may be associated with some embodiments of the therapy system of FIG. 1;

FIG. 15B is a detail end view of a portion of the therapy unit of FIG. 15A illustrating additional details that may be associated with some embodiments of the therapy system of FIG. 1;

FIG. 16 is a perspective view of a first connecting rod of the therapy unit illustrating additional details that may be associated with some embodiments of the therapy system of FIG. 1;

FIG. 17 is a side view of the first connecting rod illustrating additional details that may be associated with some embodiments of the therapy system of FIG. 1;

FIG. 18 is a front view of the first connecting rod and the second connecting rod illustrating additional details that may be associated with some embodiments of the therapy system f FIG. 1;

FIG. 19 is an end view of the first connecting rod and the second connecting rod illustrating additional details that may be associated with some embodiments of the therapy system of FIG. 1;

FIG. 20 is a front view of the first connecting rod and the second connecting rod illustrating additional details that may be used with the therapy system of FIG. 1; and

FIG. 21 is a side view of the first connecting rod and the second connecting rod illustrating additional details that may be associated with the therapy system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

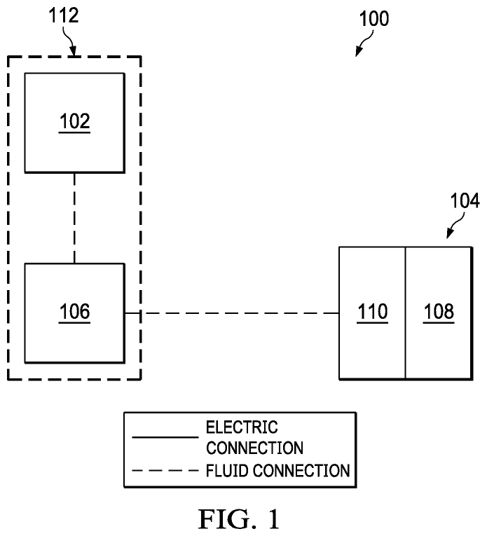

FIG. 1 is a simplified functional block diagram of an example embodiment of a therapy system 100 that can provide negative-pressure therapy to a tissue site in accordance with this specification. The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. As illustrated in the example of FIG. 1, the dressing 104 may comprise or consist essentially of a tissue interface 108, a cover 110, or both in some embodiments.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104. For example, such a dressing interface may be a SENSAT.R.A.C.™ Pad available from Kinetic Concepts, Inc. of San Antonio, Tex.

The therapy system 100 may also include a regulator or controller. Additionally, the therapy system 100 may include sensors to measure operating parameters and provide feedback signals to the controller indicative of the operating parameters. For example, the therapy system 100 may include a first sensor and a second sensor coupled to a controller.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the container 106 and other components into a therapy unit 112.

In general, components of the therapy system 100 may be coupled directly or indirectly. For example, the negative-pressure source 102 may be directly coupled to the container 106 and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to a controller and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

The tissue interface 108 can be generally adapted to partially or fully contact a tissue site. The tissue interface 108 may take many forms, and may have many sizes, shapes, or thicknesses, depending on a variety of factors, such as the type of treatment being implemented or the nature and size of a tissue site. For example, the size and shape of the tissue interface 108 may be adapted to the contours of deep and irregular shaped tissue sites. Any or all of the surfaces of the tissue interface 108 may have an uneven, coarse, or jagged profile.

In some embodiments, the tissue interface 108 may comprise or consist essentially of a manifold. A manifold in this context may comprise or consist essentially of a means for collecting or distributing fluid across the tissue interface 108 under pressure. For example, a manifold may be adapted to receive negative pressure from a source and distribute negative pressure through multiple apertures across the tissue interface 108, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 108 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 108 may also vary according to needs of a prescribed therapy. The 25% compression load deflection of the tissue interface 108 may be at least 0.35 pounds per square inch, and the 65% compression load deflection may be at least 0.43 pounds per square inch. In some embodiments, the tensile strength of the tissue interface 108 may be at least 10 pounds per square inch. The tissue interface 108 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 108 may be reticulated polyurethane foam such as found in GRANUFOAM™ dressing or V.A.C. VERAFLO™ dressing, both available from Kinetic Concepts, Inc. of San Antonio, Tex..

The thickness of the tissue interface 108 may also vary according to needs of a prescribed therapy. For example, the thickness of the tissue interface may be decreased to reduce tension on peripheral tissue. The thickness of the tissue interface 108 can also affect the conformability of the tissue interface 108. In some embodiments, a thickness in a range of about 5 millimeters to 10 millimeters may be suitable.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex.. Other hydrophilic foams may include those made from polyether. Other foams that may exhibit hydrophilic characteristics include hydrophobic foams that have been treated or coated to provide hydrophilicity.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 110 may comprise INSPIRE 2301 having an MVTR (upright cup technique) of 2600 g/m²/24 hours and a thickness of about 30 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, for example, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 108 may be placed within, over, on, or otherwise proximate to a tissue site. If the tissue site is a wound, for example, the tissue interface 108 may partially or completely fill the wound, or it may be placed over the wound. The cover 110 may be placed over the tissue interface 108 and sealed to an attachment surface near a tissue site. For example, the cover 110 may be sealed to undamaged epidermis peripheral to a tissue site. Thus, the dressing 104 can provide a sealed therapeutic environment proximate to a tissue site, substantially isolated from the external environment, and the negative-pressure source 102 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 108 in the sealed therapeutic environment can induce macro-strain and micro-strain in the tissue site. Negative pressure can also remove exudate and other fluid from a tissue site, which can be collected in container 106.

Some therapy units can be portable and operate independently of an electrical connection. A portable therapy unit may use a mechanical assembly to develop a negative-pressure. In some cases, the mechanical assembly may be a consumable component capable of being operated only once. For example, the mechanical assembly of a portable therapy unit may be operated a single time by a user to charge the therapy unit. As used herein, charging a therapy unit can refer to the operation of the therapy unit to generate a negative pressure in an associated dressing, such as the dressing 104. If the mechanical assembly is consumable, the therapy unit may only be charged a single time. If a leak occurs in a dressing or elsewhere in the system, the therapy unit may reach its operational limit prior to its expected operational limit. For example, if the therapy unit is expected to provide negative pressure for an hour, and the mechanical assembly is consumable, a leak may prevent the therapy unit from providing negative-pressure therapy for the expected hour.

Some mechanical assemblies used to charge a portable therapy device may not be consumable, allowing the portable therapy unit to be charged multiple times. A non-consumable assembly permits the therapy unit to accommodate a leak within the therapy system by being charged a second or third time during negative-pressure therapy. However, many non-consumable assemblies require use of a component that is separable from the therapy unit. A separable component may become lost, preventing the therapy unit from reaching its operational limit. Some therapy units provide non-separable components associated with a non-consumable assembly, permitting the therapy unit to accommodate leaks within a system. However, the cost and complexity associated with such non-separable components has presented a significant long-held need that has not been suitably addressed in the art.

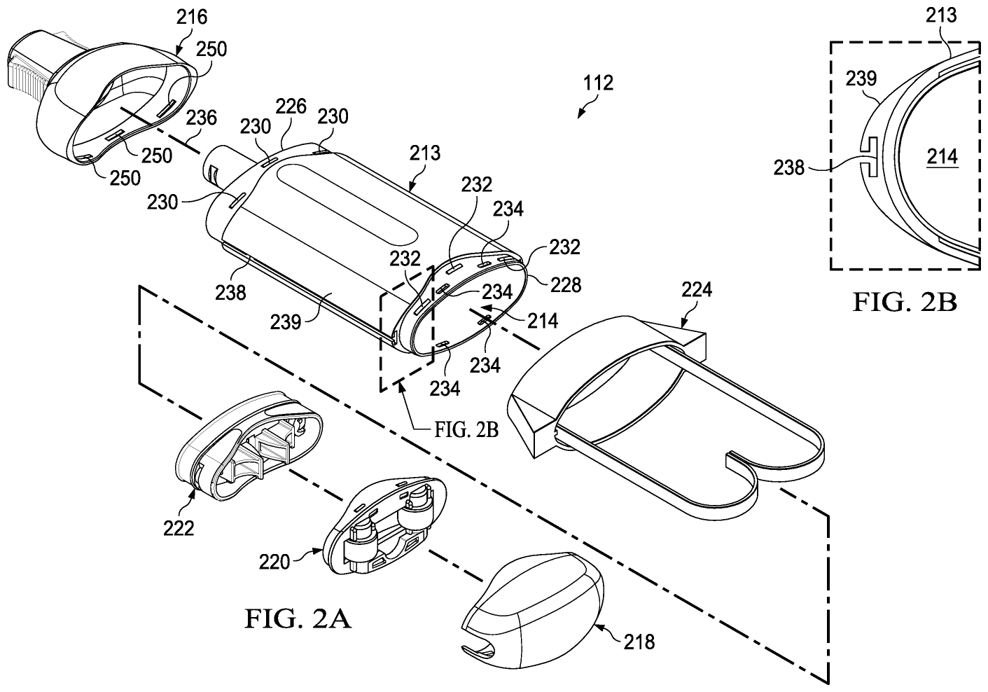

FIG. 2A is a perspective exploded view of the therapy unit 112 illustrating additional details that may be associated with some example embodiments of the therapy system 100 of FIG. 1. The therapy unit 112 can be portable, manually operated, and charged more than once. The therapy unit 112 can include housing 213 forming a suction chamber 214, a front cap 216 and an end cap 218. The therapy unit 112 can further include a spring assembly 220, a head, such as a piston assembly 222, and a charging assembly 224.

The housing 213 may be generally tubular having a non-circular sidewall defining the suction chamber 214. The housing 213 may have a first end 226, a second end 228, and an axis 236. The housing 213 can have a non-circular cross-sectional shape with respect to a transverse plane to the axis 236. In other examples, the cross-sectional shape of the housing 213 may have other types of geometric configurations for example, elliptical, rectangular, triangular, ovoid, amorphous, etc. The housing 213 may be fabricated from a rigid polymer adapted to maintain the external shape of the housing 213 if a negative pressure is developed in the suction chamber 214. In some embodiments, a sidewall of the housing 213 forming the non-circular cross-sectional shape may be transparent, permitting visual inspection of the quantity and quality of wound exudates contained within the suction chamber 214. In other embodiments, the sidewall of the housing 213 may be non-transparent. The sidewall of the housing 213 may include a transparent portion that is less than the entirety of the housing 213, forming an inspection window.

The suction chamber 214 can be a void space within the housing 213. In some embodiments, the suction chamber 214 can serve as a repository for the storage of fluids and exudates drawn from a tissue site during negative-pressure therapy. The suction chamber 214 may also serve as a pump chamber through which the therapy unit 112 can generate a negative pressure with which to draw fluids from a tissue site.

Notches 230 may be formed in the first end 226 of the housing 213, and notches 232 may be formed in the second end 228 of the housing 213. The notches 230 may facilitate coupling of the front cap 216 to the first end 226, and the notches 232 may facilitate coupling of the end cap 218 to the second end 228. Notches 234 may also be formed in the second end 228. The notches 234 may facilitate coupling of the spring assembly 220 to the second end 228 of the housing 213.

The front cap 216 may be detachably secured to the first end 226 of the housing 213. For example, the front cap 216 may have one or more keys 250 projecting from an inner or outer surface of the front cap 216 that mate with the notches 230 of the housing 213. The keys 250 may be inserted into the notches 230 to secure the front cap 216 to the housing 213.

FIG. 2B is a detail end view of a portion of the therapy unit of FIG. 2A illustrating additional details that may be associated with some example embodiments of the therapy system of FIG. 1. The housing 213 can have grooves. The grooves can comprise one or more slider channels 238. The slider channels 238 are formed in the sidewall of the housing 213 and extend from the first end 226 to the second end 228 parallel to the axis 236. The slider channels 238 may be formed in protuberances 239 that protrude from the sidewall of the housing 213 parallel to the axis 236. As illustrated in the detail view of FIG. 2, the slider channels 238 can be T-shaped having a vertical component of the T extending from an exterior surface of the housing 213 toward the suction chamber 214 and a horizontal component of the T disposed within and tangential to the sidewall of the housing 213. In some embodiments, a lubrication may be provided within the slider channels 238. While illustrated on opposite vertices of the ellipse forming the housing 213, the slider channels 238 and the first connecting rod 604 and the second connecting rod 606 can be positioned at other locations of the housing 213.

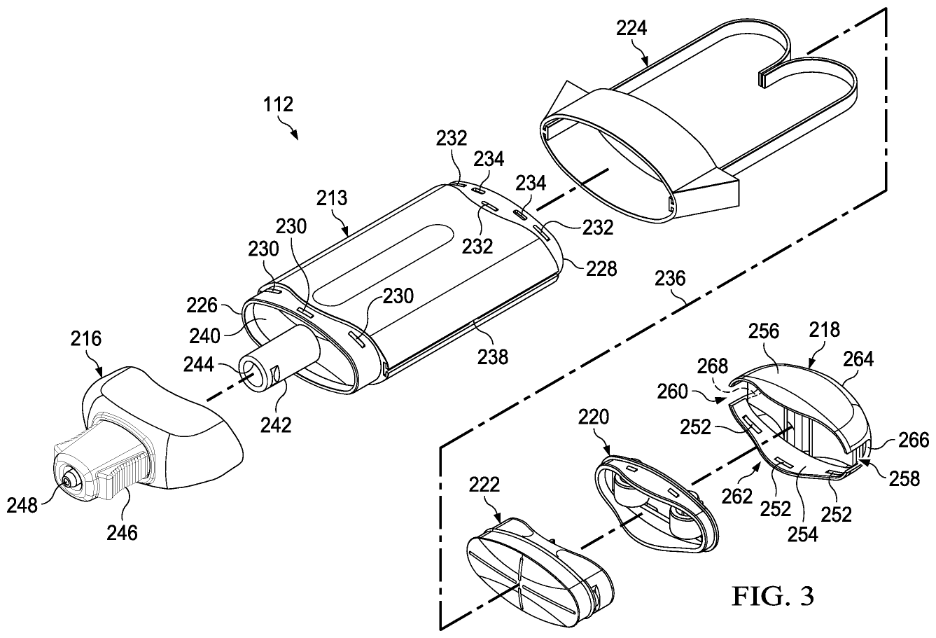

FIG. 3 is a perspective exploded view of the therapy unit 112 illustrating additional details that may be associated with some example embodiments of the therapy system 100 of FIG. 1. The housing 213 may include an end wall 240 coupled to the first end 226. The end wall 240 of the housing can include a conduit 242 or other extension structure. The conduit 242 includes a conduit lumen 244 in fluid communication with the suction chamber 214 through the end wall 240. A user-controlled valve may be disposed in the conduit lumen 244 to open or close fluid communication with the suction chamber 214. Fluid communication may also be controlled automatically by the coupling and/or decoupling of device components to the therapy unit 112.

A fitting housing 246 may be coupled to the front cap 216, enclosing a fitting 248, such as a nozzle, fluidly coupled to the suction chamber 214 through the conduit lumen 244 if the front cap 216 is coupled to the housing 213. The fitting 248 may fluidly couple the therapy unit 112 to another component of the therapy system 100, for example, a conduit or a connector on the cover 110 of the dressing 104. The fitting housing 246 may be removably coupled to the front cap 216. In other embodiments, the fitting housing 246 may be integrally formed with the front cap 216 or otherwise configured not to be detached once coupled to the front cap 216.

The end cap 218 may be detachably secured to the second end 228 of the housing 213. For example, the end cap 218 may have one or more keys 252 projecting from an inner or outer surface of the end cap 218 that mate with the notches 232 of the housing 213. The keys 252 may be inserted into the notches 232 to secure the end cap 218 to the housing 213. The end cap 218 may define an interior space 254 bordered by an exterior wall 256. The end cap 218 may have a first side 258 and a second side 260. The end cap 218 may have reflection symmetry about the axis 236, having a generally elliptical cross-sectional shape with a center located at the axis 236. The exterior wall 256 may be contoured so that the cross-section does not form a perfect ellipse. The end cap 218 may have a first end 262 and a second end 264. The first end 262 may have an opening to receive the second end 228 of the housing 213. The second end 264 may be closed, preventing movement of substances across the exterior wall 256 into the interior space 254. The exterior wall 256 may form a convex exterior surface over the second end 264. The exterior wall 256 includes a first rod opening 266 and a second rod opening 268. The first rod opening 266 and the second rod opening 268 may be located at opposite vertices of the major access at the first end 262 of the end cap 218 in the first side 258 and the second side 260, respectively. The first rod opening 266 and the second rod opening 268 extend a portion of a distance from the first end 262 toward the second end 264. The first rod opening 266 and the second rod opening 268 may each have a width perpendicular to the axis 236.

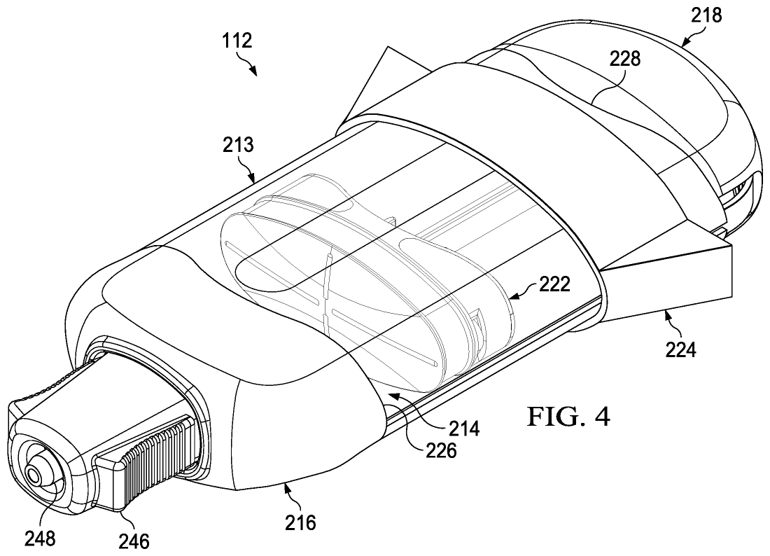

FIG. 4 is a perspective view of the therapy unit 112 illustrating additional details that may be associated with some example embodiments of the therapy system 100 of FIG. 1. As assembled, the front cap 216 may be coupled to the first end 226 of the housing 213. The piston assembly 222 may be disposed within the suction chamber 214 and operatively coupled to the spring assembly 220 (not shown) and the charging assembly 224. The spring assembly 220 may be disposed proximate the second end 228 of the housing 213. The end cap 218 may be coupled to the second end 228 of the housing 213 and operatively coupled to the charging assembly 224 for charging and/or recharging of the therapy unit 112.

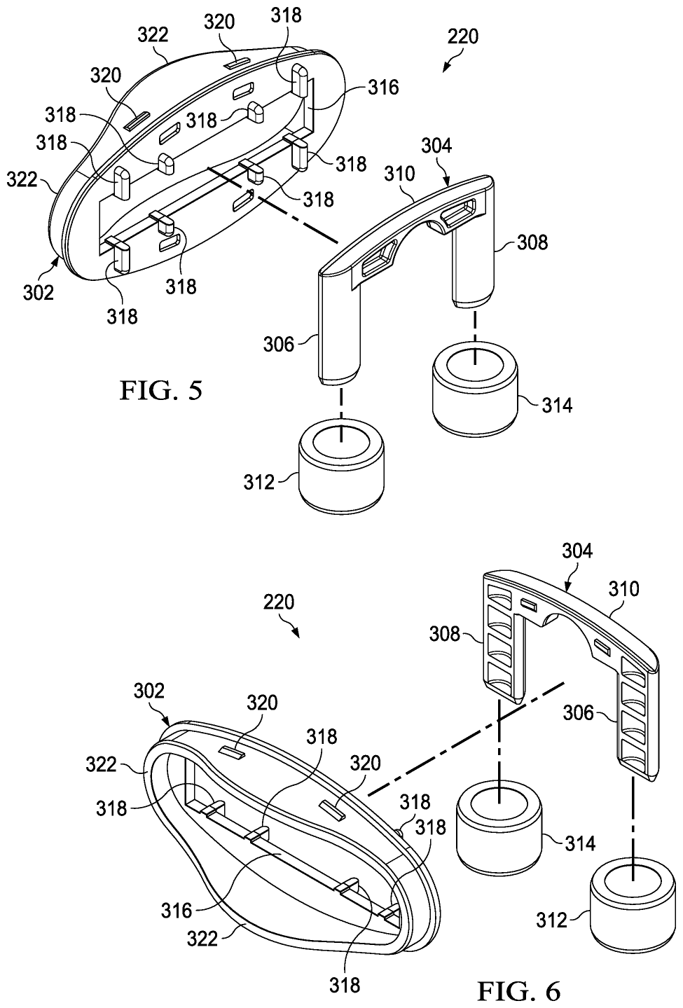

FIG. 5 is a perspective top assembly view of the spring assembly 220 and FIG. 6 is a perspective bottom assembly view of the spring assembly 220 illustrating additional details that may be associated with some example embodiments of the therapy system 100 of FIG. 1. The spring assembly 220 includes a spring carrier 302 and a spring retainer 304. The spring retainer 304 can be U-shaped having a first vertical rail 306 and a second vertical rail 308 joined by a beam 310. A first end of the first vertical rail 306 may be coupled to a first end of the beam 310. The angle formed by the first vertical rail 306 and the beam 310 may be a right angle. In other embodiments, the angle formed by the first vertical rail 306 and the beam 310 may be greater than or less than ninety degrees. A first end of the second vertical rail 308 may be coupled to a second end of the beam 310. The second end of the beam is opposite the first end of the beam 310. As used herein, opposite refers a position at the other end, side, or corner of an object or located across from an object. The angle formed by the second vertical rail 308 and the beam 310 may be a right angle. In other embodiments, the angle formed by the second vertical rail 308 and the beam 310 may be greater than or less than ninety degrees.

A first bushing 312 is disposed on the first vertical rail 306, and a second bushing 314 is disposed on the second vertical rail 308. A substantially constant force spring (not shown) may be coupled to each of the first bushing 312 and the second bushing 314. The spring carrier 302 includes a central opening 316. One or more ridges 318 may be disposed around the central opening 316. The spring retainer 304 may be coupled to the spring carrier 302. If the spring retainer 304 is coupled to the spring carrier 302, the central opening 316 can receive the first bushing 312 and the second bushing 314 and the constant-force springs associated with each bushing. Generally, a gap may exist between the first bushing 312 and the second bushing 314 in the central opening 316 if the spring retainer 304 is coupled to the spring carrier 302. The ridges 318 can be positioned to limit the movement of the first bushing 312 and its associated spring, and the second bushing 314 and its associated spring. The ridges 318 can reduce deflections or deformations of the springs during operation of the therapy unit 112. The spring carrier 302 may also include one or more keys 320 disposed around an exterior surface of the spring carrier 302. The keys 320 may be configured and located to mate with the notches 234 in the second end 228 of the housing 213. The spring carrier 302 may also have a distal edge 322. The distal edge 322 of the spring carrier 302 may have a curved, non-planar configuration.

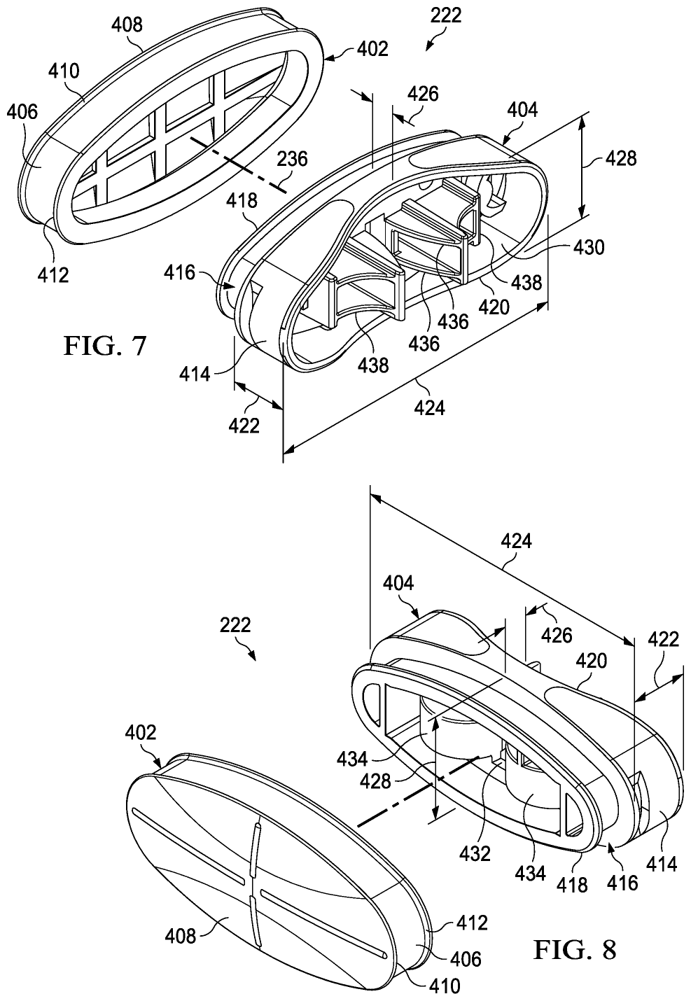

FIG. 7 is a perspective top assembly view of the piston assembly 222 and FIG. 8 is a perspective bottom assembly view of the piston assembly 222 illustrating additional details that may be associated with some example embodiments of the therapy system 100 of FIG. 1. The piston assembly 222 can include a piston seal 402 and a piston 404. The piston seal 402 may have a non-circular, elliptical cross-sectional shape with respect to the axis 236. In other embodiments, the piston seal 402 may have other shapes. The piston seal 402 may comprise a side wall 406 and an end wall 408. The side wall 406 of the piston seal 402 may include a first end wall flange 410 coupled to a first end of the side wall 406 adjacent to the end wall 408, and a second end wall flange 412 coupled to a second end of the side wall 406, the second end of the side wall 406 being opposite the first end of the side wall 406. The first end wall flange 410 and the second end wall flange 412 may have an exterior dimension greater than an exterior dimension of the side wall 406.

The piston 404 may be an elliptical frame with a side wall 414. The piston 404 may have a first end configured to be adjacent to the piston seal 402 and a second end opposite the first end. The first end of the side wall 414 may comprise a recess 416 and a raised edge or flange 418 configured to form a complementary fit with the piston seal 402. The second end of the side wall may have a perimeter edge 420. The perimeter edge 420 of the side wall 414 may have a curved, non-planar configuration.

The piston seal 402 may detachably couple to the piston 404. In some embodiments, the piston seal 402 and the piston 404 may be integrally formed. The piston 404 and the piston seal 402 may have a variable longitudinal length around the perimeter of the piston 404 and the piston seal 402. In some instances, a variable longitudinal length may provide additional stability to the piston seal 402 and the piston 404. In some examples, a length along a section of the perimeter of the piston 404 may be related to the transverse dimension intersecting: a) that length of the perimeter; and b) the axis 236 of the piston seal 402 and/or piston 404. A lateral longitudinal surface of the piston 404 may have a longitudinal length 422. A suitable longitudinal length 422 can be determined based on the width 424 of the piston 404 relative to a height of the suction chamber 214 (corresponding to the increased width and reduced height of the suction chamber 214). In comparison, a superior longitudinal surface of the piston 404 may have a longitudinal length 426 that is less than the longitudinal length 422 of the lateral longitudinal surface from the reduced height 428 of the piston 404. The piston 404 may also comprise a central opening 430. The central opening 430 may provide passage of distal ends of one or more constant force springs.

The piston 404 may further include a pair of retaining structures 432 disposed inboard of and coupled to the side wall 414. Between the central opening 430 and the retaining structures 432 are curved support surfaces 434. The curved support surfaces 434 can provide a substantial surface area to distribute forces. The piston 404 may also include one or more convex supports 436 adjacent to the central opening 430. The convex supports 436 may have a curved length. The convex supports 436 may also include a concave region 438.

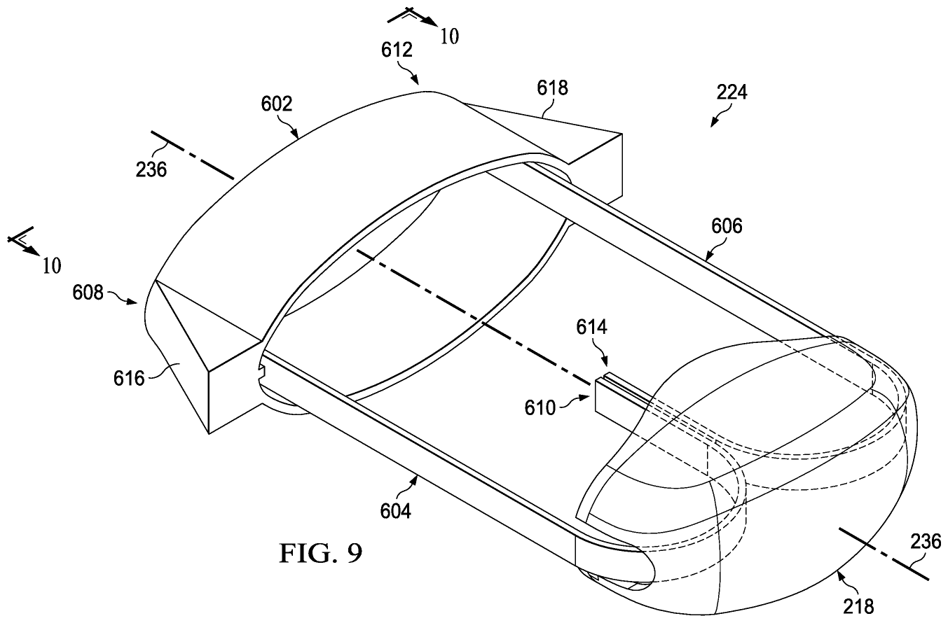

FIG. 9 is a perspective view of the charging assembly 224 illustrating additional details that may be associated with the therapy system 100 of FIG. 1. The charging assembly 224 can include a tensioner, such as a slider 602 and at least connector. The charging assembly 224 of FIG. 9 includes two connectors: a first connecting rod 604 and a second connecting rod 606. The first connecting rod 604 includes a first end 608 and a second end 610, and the second connecting rod 606 includes a first end 612 and a second end 614. The first end 608 of the first connecting rod 604 can be coupled to the slider 602. Similarly, the first end 612 of the second connecting rod 606 can be coupled to the slider 602. The first connecting rod 604 and the second connecting rod 606 extend from the slider 602 and terminate at the second end 610 and the second end 614, respectively.

In some embodiments, the slider 602 may comprise two components. For example, the slider 602 may comprise two elements, each configured to be slidingly coupled to a side of the housing 213. The slider 602 can also be formed into a ring. For example, the slider 602 may be a tubular body formed into a circular ring. The slider 602 may also have an elliptical shape. In some embodiments, the slider 602 may be molded to conform to the exterior shape of the housing 213. The slider 602 can include a finger location. For example, the slider 602 includes a first finger location 616 and a second finger location 618. In some embodiments, the slider 602 may have a rigidity that is greater than the rigidity of the first connecting rod 604 and the second connecting rod 606. Preferably, the rigidity of the slider 602 may be selected to prevent binding of the slider 602 on the housing 213 during operation of the slider 602. The first connecting rod 604 and the second connecting rod 606 may be manufactured separately from the slider 602 and coupled to the slider 602. In other embodiments, the first connecting rod 604, the second connecting rod 606, and the slider 602 may be formed from the same material. In still other embodiments, the first connecting rod 604, the second connecting rod 606, and the slider 602 may be molded or over molded to each other. In some embodiments, the slider 602 may be partially or fully transparent. If the slider 602 is substantially transparent, the contents in the suction chamber 214 may be viewable. Alternatively, graphics, such as logos, measurement markers or other similarly visible elements may be provided on the slider 602.

The first connecting rod 604 and the second connecting rod 606 may be formed from an injection molded polymer. For example, the polymer may be a thermoplastic polyurethane (“TPU”), a thermoplastic elastomer (“TPE”), or silicone. Preferably, the first connecting rod 604 and the second connecting rod 606 may have a Shore hardness rating of about 60 Shore A to about 90 Shore A. The first connecting rod 604 and the second connecting rod 606 may minimally compress along a length of the first connecting rod 604 and the second connecting rod 606 from the first end 608 and the first end 612 to the second end 610 and the second end 614, respectively. In some embodiments, the first connecting rod 604 and the second connecting rod 606 may each have a length between about 100 mm and about 105 mm. In other embodiments, a length of each of the first connecting rod 604 and the second connecting rod 606 may be adjusted based on an axial length of the suction chamber 214. For example, each of the first connecting rod 604 and the second connecting rod 606 may have a length that extends from the first end 226 of the housing 213 out of the suction chamber 214, and beyond the end cap 218. The length of each of the first connecting rod 604 and the second connecting rod 606 may be selected to permit the piston assembly 222 and the slider 602 to travel a full length of the suction chamber 214 so that the piston assembly 222 may be adjacent to the end wall 240 in a first position and adjacent to the spring assembly 220 in a second position. The first connecting rod 604 and the second connecting rod 606 may be considered solid bodies. For example, a force applied to the first end 608 of the first connecting rod 604 causing translation of the first end 608 parallel to the axis 236 can result in translation of the second end 610 of the first connecting rod 604 of an equal magnitude. Similarly, a force applied to the first end 612 of the second connecting rod 606 causing translation of the first end 612 parallel to the axis 236 can result in translation of the second end 614 of the second connecting rod 606 of an equal magnitude. The first connecting rod 604 may also be flexible. For example, the second end 610 may be fixed and the first end 608 can be brought to meet the second end 610 of the first connecting rod 604 without causing failure of the first connecting rod 604. Similarly, the second end 614 of the second connecting rod 606 may be fixed and the first end 612 can be brought to meet the second end 614 of the second connecting rod 606 without causing failure of the second connecting rod 606.

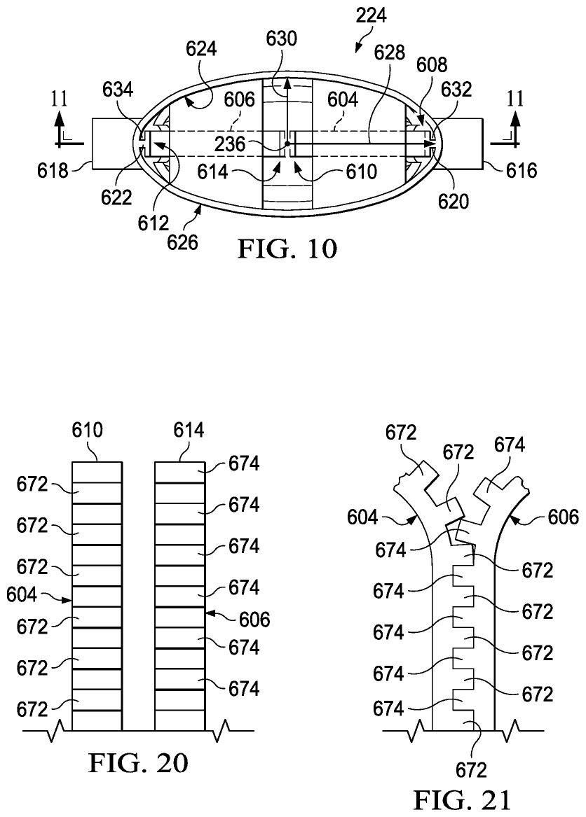

FIG. 10 is an end view of the end cap 218 and the charging assembly 224 illustrating additional details that may be associated with some embodiments of the therapy system 100 of FIG. 1. The slider 602 may be an elliptical ring. The slider 602 may have an inner surface 624 and an outer surface 626. The slider 602 may have a semi-major axis 628 and a semi-minor axis 630. The charging assembly 224 may include a first key 620 and a second key 622. The first key 620 and the second key 622 may be located on and coupled to the inner surface 624 at the vertex of the semi-major axis 628 of the slider 602. The first end 608 of the first connecting rod 604 may be coupled to the first key 620, and the first end 612 of the second connecting rod 606 may be coupled to the second key 622. The first key 620 and the second key 622 may have a width that is less than a width of the first connecting rod 604 and the second connecting rod 606, creating a gap 632 between the first connecting rod 604 and the inner surface 624 and a gap 634 between the second connecting rod 606 and the inner surface 624.

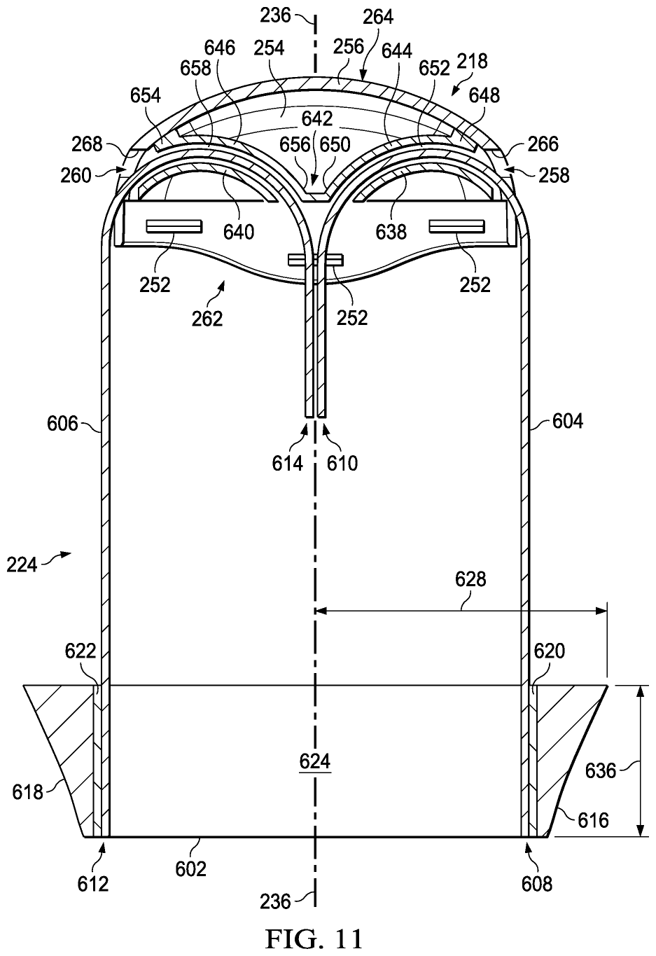

FIG. 11 is a section view of the end cap 218 and the charging assembly 224 taken along line 11-11 of FIG. 10 illustrating additional details that may be associated with some embodiments of the therapy system 100 of FIG. 1. The slider 602 may have a height 636. In some embodiments, the height 636 may be between about 35 mm and about 45 mm. The height 636 may be less than a length of the first connecting rod 604 and the second connecting rod 606. The first key 620 and the second key 622 may have a height equal to the height 636. In other embodiments, the first key 620 and the second key 622 may have a height less than the height 636. The first connecting rod 604 may be coupled to the first key 620 over the length of the first key 620, and the second connecting rod 606 may be coupled to the second key 622 over the length of the second key 622. The first finger location 616 and the second finger location 618 may have a wedge shape. A narrow end of the wedge shape may be proximate to a first end of the slider 602 and the first finger location 616 and the second finger location 618 may extend the height 636. In other embodiments, the narrow end of the wedge shape may be proximate to a second end of the slider 602. The first finger location 616 and the second finger location 618 can have other shapes, including spherical, square, ovoid, amorphous, etc. In some embodiments, the second end 610 of the first connecting rod 604 may be coupled to the second end 614 of the second connecting rod 606.

The end cap 218 may have one or more interior walls. For example, the end cap 218 may include a first wall 638, a second wall 640, and a third wall 642. The first wall 638 extends radially relative to the axis 236 from the axis 236 toward the first side 258. The first wall 638 may have a concave surface facing the first end 262 and a convex surface facing the second end 264. In some embodiments, the first wall 638 may have a radius of curvature of about 9 mm to about 10 mm. The first wall 638 can be positioned between the first end 262 and the second end 264. Similarly, the second wall 640 extends radially relative to the axis 236 from the axis 236 toward the second side 260. The second wall 640 may have a concave surface facing the first end 262 and a convex surface facing the second end 264. The second wall 640 can be positioned between the first end 262 and the second end 264. The first wall 638 and the second wall 640 may be at about the same axial distance between the first end 262 and the second end 264.

The third wall 642 can be disposed within the interior space 254 between the first wall 638 and the second wall 640 and the second end 264. The third wall 642 may have a first portion 644 between the first side 258 and the axis 236 and a second portion 646 between the second side 260 and the axis 236. The first portion 644 may have a concave surface facing the first wall 638 and a convex surface facing the second end 264. A first end 648 of the first portion 644 may be proximate to the first rod opening 266. A second end 650 of the first portion 644 may be proximate to the axis 236. A tangential plane of the concave surface of the first portion 644 may be parallel to the axis 236 at the second end 650. Preferably, a tangential plane of the concave surface of the first portion 644 may be parallel to the axis 236 at the first end 648. The concave surface of the first portion 644 can be semi-circular. In some embodiments, the concave surface may be less than a full semi-circle. The first wall 638 and the first portion 644 form a channel 652 between the convex surface of the first wall 638 and the concave surface of the first portion 644 of the third wall 642. In some embodiments, the channel 652 may have a depth between the first wall 638 and the first portion 644 of the third wall 642 that is about a thickness of the first connecting rod 604 plus between about 2 mm and about 3 mm.

The second portion 646 may have a concave surface facing the second wall 640 and a convex surface facing the second end 264. A first end 654 of the second portion 646 may be proximate to the second rod opening 268. A second end 656 of the second portion 646 may be proximate to the axis 236. A tangential plane of the concave surface of the second portion 646 may be parallel to the axis 236 at the second end 656. Preferably, a tangential plane of the concave surface of the second portion 646 may be parallel to the axis 236 at the first end 654. The concave surface of the second portion 646 can be semi-circular. In some embodiments, the concave surface may be less than a full semi-circle. The second wall 640 and the second portion 646 form a channel 658 between the convex surface of the second wall 640 and the concave surface of the second portion 646 of the third wall 642. In some embodiments, the channel 658 may have a depth between the second wall 640 and the second portion 646 of the third wall 642 that is about a thickness of the second connecting rod 606 plus between about 2 mm and about 3 mm. The second end 650 of the first portion 644 may be coupled to the second end 656 of the second portion 646. In other embodiments, the second end 650 of the first portion 644 and the second end 656 of the second portion 646 may be free.

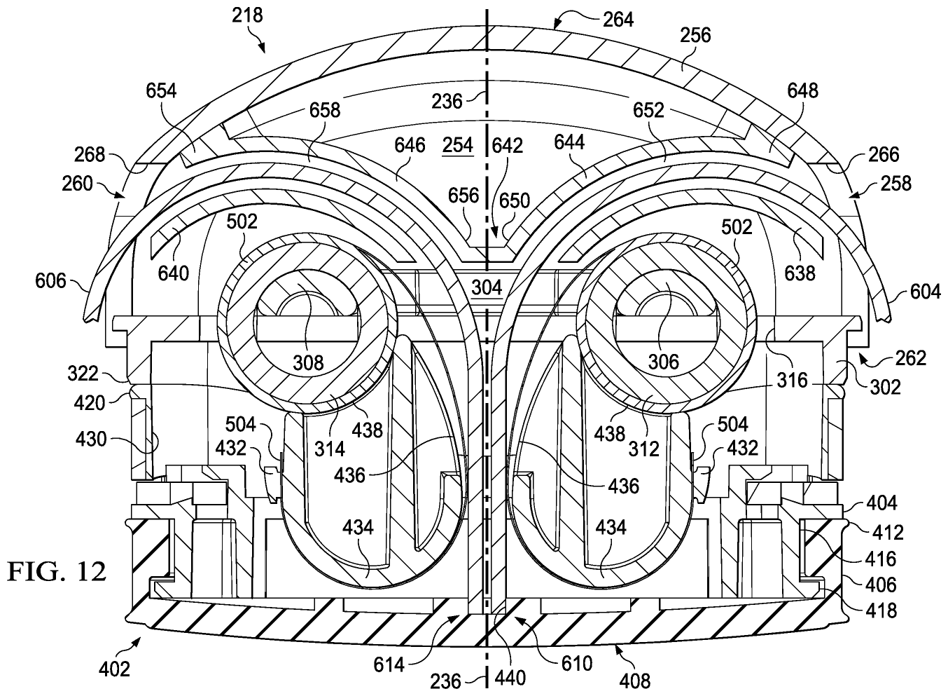

FIG. 12 is an assembled sectional view of the end cap 218, the spring assembly 220, the piston assembly 222, and the charging assembly 224 illustrating additional details that may be associated with the therapy system 100 of FIG. 1. The spring retainer 304 may be coupled to the spring carrier 302. The first bushing 312 and the second bushing 314 may be mounted on the first vertical rail 306 and the second vertical rail 308 of the spring retainer 304 and be at least partially inserted into the central opening 316. Constant-force springs 502 may be mounted to the first bushing 312 and the second bushing 314. Each constant-force spring 502 may have a free end 504 extending through the central opening 316. A constant force spring may be a spring for which the force the spring exerts over its range of motion is generally constant. The constant-force spring 502 may be constructed from a rolled ribbon of spring steel or similar having a first end secured to a bushing, for example, the first bushing 312, and the free end 504. The free end 504 can be distally extended and attached to the piston assembly 222. In some embodiments, other springs may be used. A spring constant of the constant-force spring 502 may be selected for a desired therapeutic pressure of the therapy unit 112.

The piston seal 402 can be brought adjacent to the piston 404 so that the flange 418 fits and secures the piston seal 402 to the piston 404. The second end wall flange 412 and the side wall 406 of the piston seal 402 may fit into the recess 416 of the piston 404. The central opening 430 may be aligned with the central opening 316. The perimeter edge 420 of the side wall 414 may have a complementary shape to the distal edge 322 of the spring carrier 302. The perimeter edge 420 may be brought proximate to and in contact with the distal edge 322.

The free ends 504 of the constant-force springs 502 may extend through the central opening 430 and are coupled to the retaining structures 432. In this particular embodiment, the retaining structures 432 are configured to be inserted into apertures provided on the free ends 504 of the constant-force springs 502 and may maintain their coupling using residual spring force that may be present in the constant-force springs 502 in a retracted position. In the retracted position, the piston assembly 222 may be proximate to or adjacent to the spring assembly 220. In some embodiments, the perimeter edge 420 may be adjacent to the distal edge 322 in the retracted position. The retaining structures 432 and the constant-force springs 502 may have a variety of other coupling configurations, for example, the retaining structures 432 may comprise posts which block displacement of the free ends 504, which can be T-shaped. The free ends 504 of the constant-force springs 502 may be passed over the curved support surfaces 434 so that a side of the ribbon contacts the curved support surface 434. The curved support surfaces 434 can push against the constant-force springs 502. In some examples, the length of the curved support surfaces 434 between the central opening 430 and the each of the retaining structures 432 may be at least one or one and a half times a width of each constant-force spring 502. In other examples, the length of each of the curved support surfaces 434 may be two, three, or four times the width of each constant-force spring 502. In some examples, the curved support surfaces 434 provide a substantial surface area to distribute the pushing forces, reducing the risk of damage to the constant-force springs 502.

The convex supports 436 adjacent to the central opening 430 may support the constant-force springs 502 as the constant-force springs 502 converge into the central opening 430. The convex supports 436 may have a curved length of at least about the width of the constant-force springs 502, but in other examples may be at least two times or three times the width of the constant-force springs 502. The concave region 438 may accommodate the coils of the constant-force spring 502 mounted on the spring carrier 302 when the piston assembly 222 is in the retracted position. In the retracted position, the first wall 638 does not interfere with rotation of the constant-force spring 502 and the first bushing 312. Similarly, the second wall 640 does not interfere with rotation of the constant-force spring 502 and the second bushing 314. Although the piston assembly 222 and the spring assembly 220 use two constant-force springs 502, in other examples, one spring, three springs, four springs, or five or more springs may be used. The number of springs, the type of springs, and the width and length of the springs may be varied, and in other examples, non-spring bias members may be used, for example, sealed pneumatic shocks.

The second end 610 of the first connecting rod 604 can be inserted into the first rod opening 266 of the end cap 218 and through the channel 652. The first rod opening 266 may have a width that is greater than a width of the first connecting rod 604. As the first connecting rod 604 is inserted into the channel 652, the interaction between the second end 610 and the channel 652 turns the first connecting rod 604 through a 180 degree arc. The second end 610 can be passed through the central opening 316, the central opening 430, between the constant-force springs 502 and coupled to the piston seal 402. The second end 610 may be inserted into a recess 440 located proximate a center of the piston seal 402 on an interior surface of the end wall 408 and bonded, welded, fastened, or otherwise coupled to the piston seal 402.

The second end 614 of the second connecting rod 606 can be inserted into the second rod opening 268 of the end cap 218 and through the channel 658. The second rod opening 268 may have a width that is greater than a width of the second connecting rod 606. As the second connecting rod 606 is inserted into the channel 658, the interaction between the second end 614 and the channel 658 turns the second connecting rod 606 through a 180 degree arc. The second end 614 can be passed through the central opening 316, the central opening 430, between the constant-force springs 502, and coupled to the piston seal 402. The second end 614 may be inserted into the recess 440 located proximate a center of the piston seal 402 on an interior surface of the end wall 408 and bonded, welded, fastened, or otherwise coupled to the piston seal 402. In some embodiments, the second end 610 of the first connecting rod 604 may be coupled to the second end 614 of the second connecting rod 606.

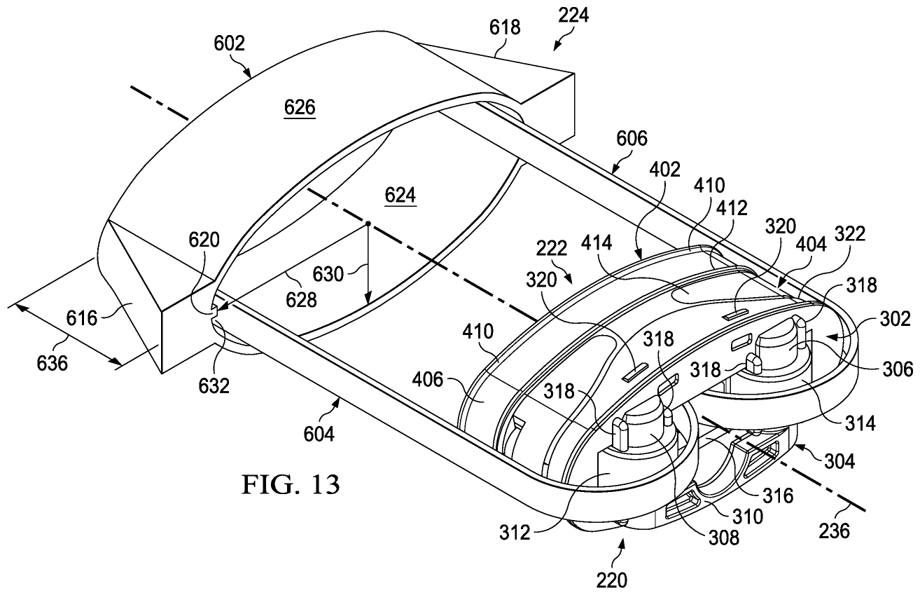

FIG. 13 is a perspective view of the spring assembly 220, the piston assembly 222, and the charging assembly 224 illustrating additional details that may be associated with the therapy system 100 of FIG. 1. The piston assembly 222 is in the retracted position so that the perimeter edge 420 of the piston 404 of the piston assembly 222 is proximate to and, in some cases, adjacent to the distal edge 322 of the spring carrier 302 of the spring assembly 220. In the retracted position, the slider 602 is axially separated from the piston assembly 222 and the spring assembly 220.

FIG. 14 is a perspective assembly view of the spring assembly 220, the piston assembly 222, and the charging assembly 224 illustrating additional details that may be associated with some embodiments of the therapy system 100 of FIG. 1. The piston assembly 222 and the charging assembly 224 are in a charged position. In the charged position, the piston assembly 222 may be axially separated from the spring assembly 220, extending the free ends 504 of the constant-force springs 502 (not shown). In the charged position, the slider 602 of the charging assembly 224 may be axially proximate to the spring assembly 220. The slider 602 and the piston assembly 222 are translationally related. For example, movement of the slider 602 in a first direction results in movement of the piston assembly 222 in a second direction. Movement of the slider 602 and the piston assembly 222 in both the first direction and the second direction is parallel to the axis 236. The first connecting rod 604 and the second connecting rod 606 translationally couple the piston assembly 222 and the slider 602.

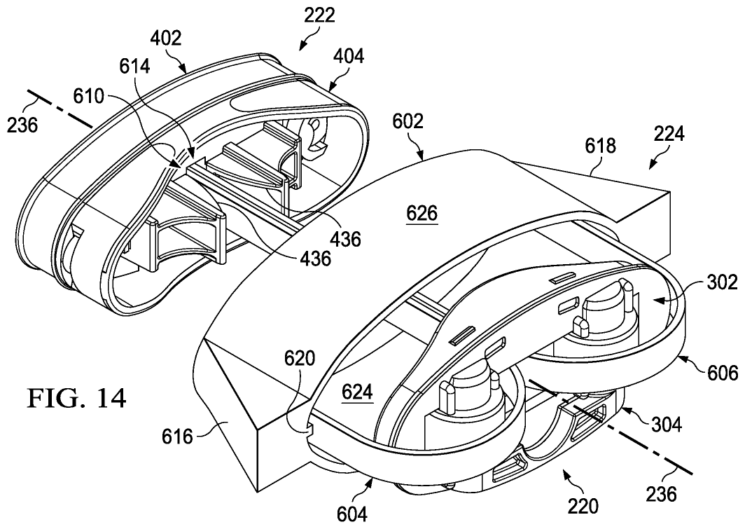

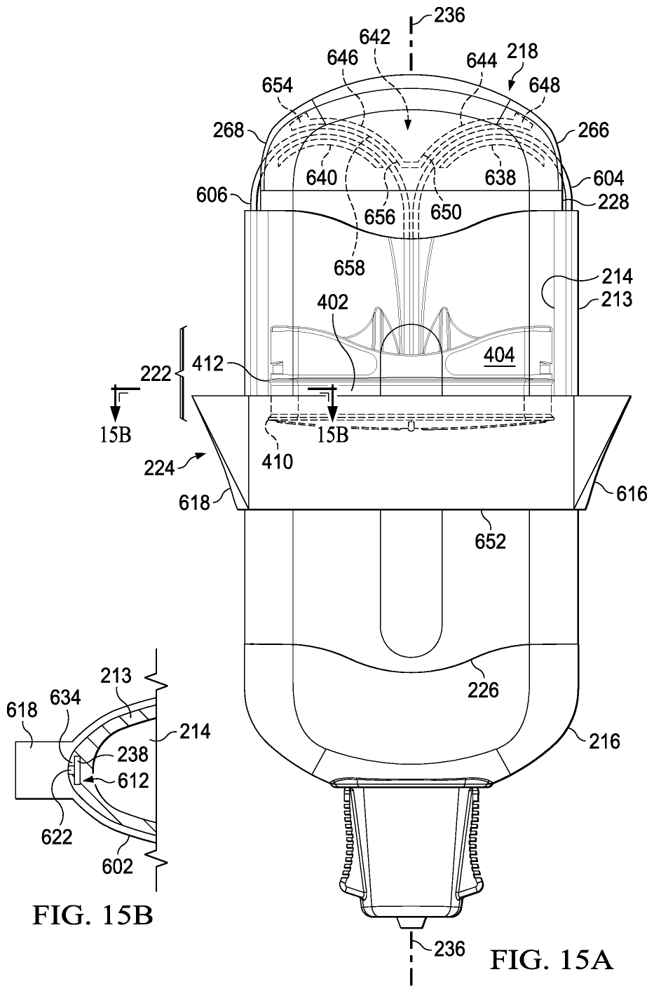

FIG. 15A is a front view of the therapy unit 112 illustrating additional details that may be associated with some embodiments of the therapy system 100 of FIG. 1. The piston assembly 222 may be configured to traverse between the first end 226 and the second end 228 of the housing 213 within the suction chamber 214 while maintaining a substantially airtight seal. As shown, the piston assembly 222 and the slider 602 are between the charged position and the retracted position. The first end wall flange 410 and the second end wall flange 412 may be in a sliding contact with the interior surface of the housing 213. The first end wall flange 410 and the second end wall flange 412 may provide a sealed contact while limiting sliding friction. The exterior surfaces of the piston seal 402 and/or the interior surfaces of the housing 213 may comprise a friction-reducing lubricant or a lubricious coating material.

The first connecting rod 604 and the second connecting rod 606 couple the slider 602 to the piston assembly 222. Sliding contact between the first connecting rod 604 and the first portion 644 of the third wall 642 and between the first connecting rod 604 and the first wall 638 can cause the first connecting rod 604 to deform as the slider 602 moves parallel to the axis 236. Similarly, sliding contact between the second connecting rod 606 and the second portion 646 of the third wall 642 and between the second connecting rod 606 and the second wall 640 can cause the second connecting rod 606 to deform as the slider 602 moves parallel to the axis 236.

FIG. 15B is a detail end view of a portion of the therapy unit of FIG. 15A illustrating additional details that may be associated with some embodiments of the therapy system of FIG. 1. The second connecting rod 606 and the second key 622 may fit into, slide through, and are captured by the slider channel 238. Similarly, the first connecting rod 604 and the first key 620 may fit into, slide through, and are captured by the slider channel 238 on an opposite side of the housing 213. The slider channels 238 can maintain an alignment of the first connecting rod 604 and the second connecting rod 606, preventing deformation of the first connecting rod 604 and the second connecting rod 606 prior to passage through the end cap 218. In some embodiments, the slider channels 238 may be lubricated. The housing 213 may also be formed from a polymer, such as silicone, having lubricating characteristics.

In some embodiments, the therapy unit 112 may be supplied pre-charged so that the therapy unit 122 can be provided with the piston assembly 222 in the charged position, having the constant-force springs 502 fully extended. The slider 602 may be axially proximate to the end cap 218 and the spring assembly 220. The therapy unit 112 may be locked into the charged position by a component, such as an activation key. The activation key can be inserted about slider 602 and can be removed and discarded if the therapy unit 112 is connected to another component. Removal of the activation key from the slider 602 permits the constant-force springs 502 to pull the piston assembly 222 toward the spring assembly 220 and the retracted position. Movement of the piston assembly 222 toward the spring assembly 220 generates a negative pressure in the suction chamber 214 between the piston assembly 222 and the first end 226 of the housing 213. The negative pressure can be communicated to the dressing 104 or other device to draw fluid into the suction chamber 214. In response to movement of the piston assembly 222 toward the retracted position, the slider 602 can slide down the housing 213 toward the first end 226 of the housing 213. In some embodiments, the housing 213 can have markings. The markings can be positioned on the exterior of the housing 213. Identification of the relative distance between the slider 602 and the markings on the housing 213 can indicate the approximate location of the piston assembly 222 within the suction chamber 214 and whether the piston assembly 222 can be returned to the charged position.

To charge the therapy unit 112, the slider 602 may be proximate to the first end 226 of the housing 213, and the piston assembly 222 may be in the retracted position. The first finger locator 616 and the second finger locater 618 may be used to move the slider 602 in a first direction toward the second end 228 of the housing 213 parallel to the axis 236. In response, the first end 608 of the first connecting rod 604 and the first end 612 of the second connecting rod 606 can be moved in the first direction toward the end cap 218, sliding the first connecting rod 604 and the second connecting rod 606 through the slider channels 238. Portions of the first connecting rod 604 and the second connecting rod 606 within the end cap 218 contact the third wall 642 and deform the first connecting rod 604 and the second connecting rod 606 through a 180 degree turn, causing the translation in the first direction to become translation of the second end 610 and the second end 614 in the second direction. Thus, as the first connecting rod 604 and the second connecting rod 606 slide through the slider channels 238, the piston assembly 222 is depressed and moved to the charged position. The slider 602 can be moved from the retracted position to the charged position each time the piston assembly 222 reaches the retracted position until the dressing 104 coupled to the therapy unit 112 is fully evacuated. Full evacuation can be indicated by the inability of the piston assembly 222 and the slider 602 to move from the charged position to the retracted position.

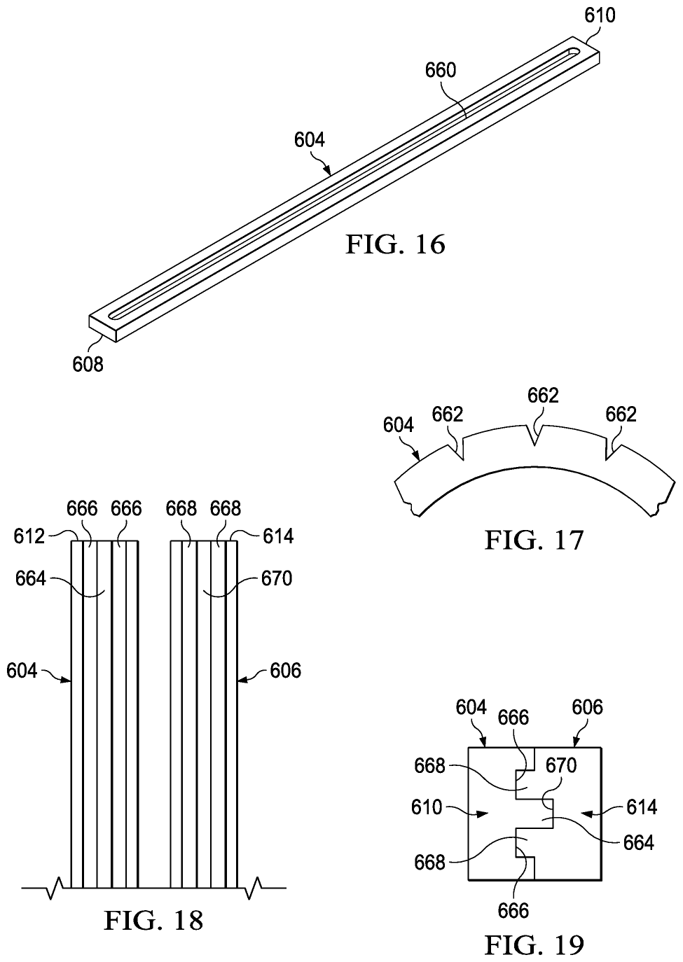

FIG. 16 is a perspective view of the first connecting rod 604 illustrating additional details that may be associated with some embodiments of the therapy system 100 of FIG. 1. In alternative embodiments, the first connecting rod 604 may include one or more grooves 660. The grooves 660 may run a length of the first connecting rod 604 from the first end 608 to the second end 610. The grooves 660 can be evenly spaced along the width of the first connecting rod 604. In some embodiments, the grooves 660 may have a depth less than a depth of the first connecting rod 604. In other embodiments, the grooves 660 may extend through the first connecting rod 604. If the grooves 660 extend through the first connecting rod 604, the portions adjacent the grooves 660 may remain joined at the end the first end 608 and the second end 610. The grooves 660 may facilitate bending of the first connecting rod 604. The second connecting rod 606 may also include a plurality of grooves 660 extending a length of the second connecting rod 606.

FIG. 17 is a side view of the first connecting rod 604 illustrating additional details that may be associated with some embodiments of the therapy system 100 of FIG. 1. In alternative embodiments, the first connecting rod 604 may include a plurality of slits 662. The slits 662 may run across a width of the first connecting rod 604 and be spaced at regular intervals. In other embodiments, the slits 662 may not be evenly spaced along the length of the first connecting rod 604. In some embodiments, the slits 662 may have a depth less than a depth of the first connecting rod 604. The slits 662 may facilitate bending of the first connecting rod 604. The second connecting rod 606 may also include a plurality of slits across a width of the second connecting rod 606.

FIG. 18 is a front view of the first connecting rod 604 and the second connecting rod 606 illustrating additional details that may be associated with some embodiments of the therapy system 100 of FIG. 1. In alternative embodiments, the first connecting rod 604 and the second connecting rod 606 may have a tongue and groove system. For example, the first connecting rod 604 may include at least one tongue 664 and at least one groove 666. Similarly, the second connecting rod 606 may include at least one tongue 668 and at least one groove 670. The at least one tongue 664 and the at least one groove 666 may run a length of the first connecting rod 604 from the first end 608 to the second end 610. Similarly, the at least one tongue 668 and the at least one groove 670 may run a length of the second connecting rod 606 from the first end 612 to the second end 614.

FIG. 19 is an end view of the first connecting rod 604 and the second connecting rod 606 of FIG. 18, illustrating additional details that may be associated with some embodiments of the therapy system 100 of FIG. 1. Viewing the second end 610 and the second end 614 of the first connecting rod 604 and the second connecting rod 606, the at least one tongue 664 of the first connecting rod 604 may fit within the at least one groove 670 of the second connecting rod 606. Similarly, the at least one tongue 668 of the second connecting rod 606 may fit within the at least one groove 666 of the first connecting rod 604. During operation of the therapy unit 112, the first connecting rod 604 and the second connecting rod 606 may be coupled to each other after passing through the end cap 218. For example, the respective tongues 664, 668 may be inserted into the respective grooves 670, 666. Coupling of the first connecting rod 604 to the second connecting rod 606 can increase the stiffness of the combined first connecting rod 604 and the second connecting rod 606.

FIG. 20 is a front view of the first connecting rod 604 and the second connecting rod 606 illustrating additional details that may be used with the therapy system 100 of FIG. 1. In alternative embodiments, the first connecting rod 604 and the second connecting rod 606 may have intermeshing cogs. For example, the first connecting rod 604 may include at least one cog 672. Similarly, the second connecting rod 606 may include at least one cog 674. The at least one cog 672 may extend a width of the first connecting rod 604. Similarly, the at least one cog 674 and the at least one groove 670 may extend a width of the second connecting rod 606. The first connecting rod 604 includes a plurality of cogs 672 evenly spaced along a length of the first connecting rod 604. The second connecting rod 606 includes a plurality of cogs 674 evenly spaced along a length of the second connecting rod 606.

FIG. 21 is a side view of the first connecting rod 604 and the second connecting rod 606 of FIG. 20 illustrating additional details that may be associated with the therapy system 100 of FIG. 1. The at least one cog 672 of the first connecting rod 604 may intermesh with the at least one cog 674 of the second connecting rod 606. During operation of the therapy unit 112, the first connecting rod 604 and the second connecting rod 606 may be coupled to each other after passing through the end cap 218. For example, the respective cogs 672, 764 may intermesh as respective surfaces of the first connecting rod 604 and the second connecting rod 606 are brought adjacent to each other while passing through the spring assembly 220. Coupling of the first connecting rod 604 to the second connecting rod 606 can increase the stiffness of the combined first connecting rod 604 and the second connecting rod 606.

The systems, apparatuses, and methods described herein may provide significant advantages. For example, therapy unit turns a need for a vertical continuously linear force provided by a device that is approximately 100 mm long and extends beyond the size of the therapy unit into a force which is contained linearly through the length of the existing therapy unit. The therapy unit removes the need for the use of an activation key as a charging means. The ease of use of the therapy unit is significantly improved. The therapy unit can eliminate the risk that the user will misplace the charging device. The therapy unit also provides a more intuitive charging system, removing barriers for compliance with therapy and adoption of the therapy unit. Furthermore, the therapy unit is comparable in cost to other therapy units without the novel and non-obvious improvements described herein.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 104, the container 106, or both may be eliminated or separated from other components for manufacture or sale. In other example configurations, the controller may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. An apparatus for charging a negative-pressure source, the apparatus comprising:
a body having a first end, a second end, a suction chamber, and a longitudinal axis;
a slider at least partially surrounding the body and configured to slide relative to the body parallel to the longitudinal axis;
a piston disposed in the suction chamber and configured to slide relative to the body parallel to the longitudinal axis;
a rod having a first end coupled to the slider and a second end coupled to the piston, the first end configured to move parallel to the longitudinal axis in a first direction and the second end configured to move parallel to the longitudinal axis in a second direction; and
an end cap coupled to the first end of the body, the end cap configured to locate at least one constant force spring and turn the rod from the first direction to the second direction.

2. The apparatus of claim 1, wherein the slider further comprises a ring.

3. The apparatus of claim 1, wherein the slider further comprises two sliders.

4. The apparatus of claim 3, wherein each of the two sliders further comprises a finger location.

5. The apparatus of claim 1, wherein the first direction is opposite the second direction.

6. The apparatus of claim 1, wherein the rod is an injection molded polymer construction.

7. The apparatus of claim 1, wherein the rod is formed from TPE or silicone.

8. The apparatus of claim 1, wherein the rod has a shore rating of about Shore 60 to about Shore 90.

9. The apparatus of claim 1, wherein the rod is configured to deform, forming a radius and transmitting a linear force through the radius.

10. The apparatus of claim 1, wherein the rod further comprises slits along a length of the rod, the slits configured to facilitate bending and a linear transmission of force.

11. The apparatus of claim 1, wherein the rod further comprises a plurality of grooves across a width of the rod.

12. The apparatus of claim 11, wherein the rod comprises two rods, each having a plurality of grooves, the plurality of grooves of each rod configured to mesh with each other, thereby coupling the two rods.

13. A system for generating negative pressure, the system comprising:
a pump housing having a first end, a second end, a chamber, and a longitudinal axis;
a tensioner at least partially surrounding the pump housing and configured to slide relative to the pump housing parallel to the longitudinal axis;
a head disposed in the chamber and configured to slide relative to the pump housing parallel to the longitudinal axis;
a connector having a first end coupled to the tensioner and a second end coupled to the head, the first end configured to move parallel to the longitudinal axis in a first direction and the second end configured to move parallel to the longitudinal axis in a second direction;
an end cap coupled to the first end of the pump housing, the end cap configured to locate at least one constant force spring and turn the connector through a 180 degree arc; and
a nozzle coupled to the second end of the pump housing and fluidly coupled to the chamber, the nozzle configured to be fluidly coupled to a tissue site.

14. The system of claim 13, wherein the tensioner is configured to move in the second direction, sliding the head in the first direction within the chamber.

15. The system of claim 13, wherein the connector is configured to deform, forming a radius and transmitting a linear force through the radius.

16. The system of claim 13, wherein the first direction is opposite the second direction.

17. The system of claim 13, wherein the pump housing further comprises:
an elliptical tube, the chamber disposed within the tube; and
at least one groove formed in an exterior of the elliptical tube, the at least one groove configured to receive the connector, the connector configured to slide relative to the elliptical tube through the groove.

18. A method for generating negative pressure, the method comprising:
providing a negative-pressure source, the negative pressure source comprising:
a body having a first end, a second end, a suction chamber, and a longitudinal axis,
a slider at least partially surrounding the body and configured to slide relative to the body parallel to the longitudinal axis,
a piston disposed in the suction chamber and configured to slide relative to the body parallel to the longitudinal axis,
a rod having a first end coupled to the slider and a second end coupled to the piston, the first end configured to move parallel to the longitudinal axis in a first direction and the second end configured to move parallel to the longitudinal axis in a second direction,
an end cap coupled to the first end of the body, the end cap configured to locate at least one constant force spring and turn the rod through a 180 degree arc, the at least one constant force spring being coupled to the end cap and the piston, and
a nozzle coupled to the second end of the body and fluidly coupled to the suction chamber, the nozzle configured to be fluidly coupled to a tissue site; and
moving the slider in the first direction, thereby moving the piston in the second direction and tensioning the constant force spring.

19. An apparatus for charging a negative-pressure source, the apparatus comprising:
a body having a first end, a second end, a suction chamber, and a longitudinal axis;
a piston disposed in the suction chamber and configured to slide relative to the body parallel to the longitudinal axis; and
a slider at least partially surrounding the body, the slider coupled to the piston and configured to move parallel to the longitudinal axis in a first direction, movement of the slider in the first direction causing the piston to move parallel to the longitudinal axis in a second direction.

20. The apparatus of claim 19, wherein the slider further comprises a ring.

21. The apparatus of claim 19, wherein the slider further comprises two sliders.

22. The apparatus of claim 19, wherein the first direction is opposite the second direction.

23. (canceled)
